(19) United States
(12) Patent Application Publication
(10) Pub. No.: US 2026/0069794 A1
(43) Pub. Date: Mar. 12, 2026

(54) REUSABLE LARGE VOLUME INJECTION AID

(71) Applicant: Janssen Biotech, Inc., Horsham, PA (US)

(72) Inventors: STEVEN VESOLE, San Bruno, CA (US); Ian SCRIMGEOUR, East Lothian (GB); DAVID MCLEOD, EDINBURGH (GB); JAMES GLENCROSS, EAST LOTHIAN (GB)

(21) Appl. No.: 19/107,857

(22) PCT Filed: Aug. 31, 2023
(86) PCT No.: PCT/IB2023/058601

§ 371 Date: Feb. 28, 2025

Related U.S. Application Data
(60) Provisional application No. 63/374,308, filed on Sep. 1, 2022.

(51) Int. Cl. A61M 5/315; A61M 5/20

(52) U.S. Cl. CPC A61M 5/3158; A61M 5/2033; A61M 2005/202; A61M 2005/2026; A61M 2005/2086

(57) ABSTRACT

An injection aid includes a housing, an actuation assembly, and a toggle. The housing can be adapted to receive a container. The container can include a medicament chamber and a plunger movable relative to the medicament chamber to dispense medicament out of the medicament chamber. The actuation assembly can be adapted to move the plunger relative to the medicament chamber. The toggle can be movable from a ready configuration to a firing configuration whereby the actuation assembly urges the plunger to move. The toggle can be movable from the firing configuration to the ready configuration whereby the plunger is fixed relative to the housing to pause dispensing the medicament. The toggle can be moveable from the ready configuration to the firing configuration to resume dispensing medicament from the medicament chamber.

### BACKGROUND

Parenteral pharmaceutical therapies delivered via needle or cannula through the skin may be delivered by a range of means for a range of dose volumes. Delivery method and dose volume depend on factors such as the target location, the volume and concentration of pharmaceutical product required.

The volume of fluid influences the method of delivery and time to deliver. At the bottom end of the volume range small doses of 5 ml and under may typically be delivered by injection over a period up to 60 seconds. At the top end of the volume range infusion therapies may deliver doses in excess of 30 ml over a period of hours. For some pharmaceutical therapies larger than 5 ml but lower than typically suited to infusion, the dose may be administered by manual injection using a larger volume container, for example a 20 ml container over a period up to 5 minutes, or even more.

These injections may be delivered using a hypodermic or subcutaneous needle. Or they may be delivered subcutaneously using a subcutaneous cannula and infusion line connected to the container. In such cases these large volumes must be delivered at a controlled rate over a moderate period. Current best practice requires a health care provider to deliver these using a manual process, applying a steady pressure to slowly delivery the dose over a period of minutes, e.g., 3-5 minutes or ever longer.

Manual administrations of this type over this period create a number of problems. It requires careful manual dexterity and control to maintain a constant controlled rate of injection. It requires all the health care providers attention and time which can reduce the efficiency of the health care provider and the clinic in which they work. The sustained pressure and manual manipulation can cause discomfort to the health care provider, such as hand cramps.

To overcome these problems this invention is for a reusable injection aid that can automatically deliver drug formulation to a patient over a set time period and at a constant flowrate. In some cases, this new device would replace the manual process altogether and deliver a robust and reliable alternative.

### SUMMARY

An injection aid includes a housing, an actuation assembly, and a toggle. The housing can be adapted to receive a container. The container can be a syringe or a cartridge.

The container can include a medicament chamber. The actuation assembly can be adapted to move a plunger relative to the medicament chamber to dispense medicament out of the medicament chamber. The toggle can be movable from a ready configuration to a firing configuration whereby the actuation assembly urges the plunger to move. The toggle can be movable from the firing configuration to the ready configuration whereby the plunger is fixed relative to the housing to pause dispensing the medicament. The toggle can be moveable from the ready configuration to the firing configuration to resume dispensing medicament from the medicament chamber.

### BRIEF DESCRIPTION OF THE DRAWINGS

The following detailed description will be better understood when read in conjunction with the appended drawings, in which there is shown in the drawings example embodiments for the purposes of illustration. It should be understood, however, that the present disclosure is not limited to the precise arrangements and instrumentalities shown. In the drawings:

FIG. 1A is a view of an injection aid with a cover in a closed position;

FIG. 1B is a perspective view of the injection aid of FIG. 1A with the cover in an open position;

FIG. 2A is a perspective view of the injection aid of FIG. 1A with a container received by the injection aid;

FIG. 2B is a perspective view of the injection aid of FIG. 1B with a container received by the injection aid;

FIG. 3A is a proximal perspective view of the injection aid of FIG. 2A with a toggle in a first position;

FIG. 3B is a proximal perspective view of the injection aid of FIG. 2A with the toggle in a second position;

FIG. 4 is an exploded perspective view of the injection aid of FIG. 2A;

FIG. 5 is an exploded perspective view of a chassis of the injection aid FIG. 2A;

FIG. 6 is an exploded perspective view of the rail of FIG. 5;

FIG. 7 is an exploded perspective view of the carriage of FIG. 4;

FIG. 8 is an exploded view of the anchor of FIG. 4;

FIG. 9 is a sectional view of the injection aid of FIG. 2A taken along a plane including axis A-A with a lock in a locked configuration;

FIG. 10 is a sectional view of the injection aid of FIG. 2A taken along a plane including axis A-A with a lock in an unlocked configuration;

FIG. 11 is a sectional view of the injection aid of FIG. 2A taken along a plane including axis B-B;

FIG. 12 is a top plan view of the injection aid of FIG. 1B with an upper base deck removed;

FIG. 13 is a perspective, sectional view of the injection aid of FIG. 1A with the housing and cap removed taken along a plane including axis C-C; and

FIGS. 14A, 14B, and 14C are perspective views of embodiments of an injection aid.

### DETAILED DESCRIPTION

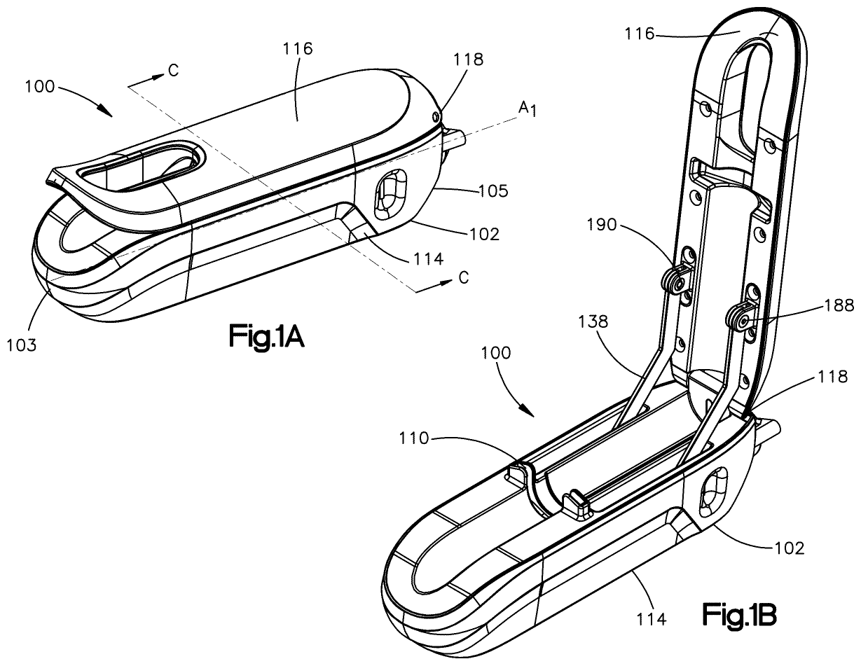

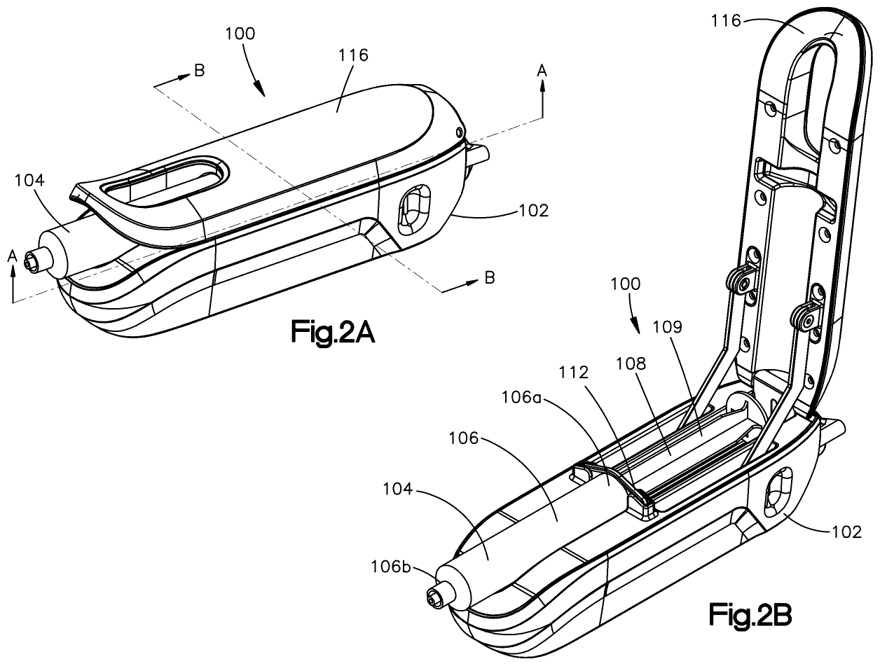

Referring initially to FIGS. 1A-2B, an injection aid 100 can provide an injection force over a selected time period to deliver medicament from a container. Injection aid 100 can provide a constant injection force over a selected time period to consistently deliver medicament from the container. Injection aid 100 can be adapted to allow the injection to be initiated, paused, and resumed, as explained below.

Injection aid 100 can be adapted to receive a container 104. Container 104 can be a syringe or cartridge. Container 104 can include a barrel 106. It is appreciated that the barrel of an injection aid typically contains a medication that is delivered from the barrel to a needle or other suitable delivery member. Barrel 106 can include a proximal end 106a and a distal end 106b. Distal end 106b can define a Luer lock connection. In this regard, it is recognized that injection aids typically include a needle, infusion set, or the like, that is attached to a barrel via a Luer lock. Alternatively, the injection training device 20 can include a needle that extends from the distal end 106b of the barrel 106, or any suitable alternative structure that extends from distal end 106b.

A seal can be disposed in barrel 106 of container 104. The seal can prevent medicament from exiting the container 104 prior to dispensing. The seal can be a plunger 108. Plunger 108 can be movable relative to barrel 106 to eject medicament from barrel 106.. In some examples, container 104 includes plunger 108 such that plunger 108 is removed from injection aid 100 when container 104 is removed from injection aid 100. In other embodiments, plunger 108 is coupled to an actuation assembly 122 such that plunger 108 is not removed when container 104 is removed from injection aid 100.

The plunger 108 can be operatively associate with a plunger rod 109. In some embodiments, the plunger 108 is fixed to plunger rod 109. In other embodiments, the plunger 108 is initially spaced from plunger rod 109 and plunger rod 109 moves into contact with the plunger 108 during a medicament dispensing event. In some embodiments, plunger rod 109 is coupled to container 104 such that plunger rod 109 is removed from injection aid 100 when container 104 is removed from injection aid 100. In other embodiments, plunger rod 109 is coupled to an actuation assembly 122 such that the plunger rod 109 is not removed when container 104 is removed from injection aid 100.

Referring to FIGS. 1A-1B, injection aid 100 can include a housing 102 adapted to receive container 104. Housing 102 can include a base 114 and a cap 116. Cap 116 can be movable relative to base 114 between an open position (FIG. 1B) and a closed position (FIG. 1A). Cap 116 can be pivotably coupled to base 114. Cap 116 can rotate about a pivot 118 relative to base 114 between the open position and the closed position. Housing 102 can include a first end 103 and a second end 105 spaced from the first end along a central axis A1. First end 103 can be a distal end and second end 105 can be a proximal end. Cap 116 can be pivotably coupled to second end 105. Housing 102 can include a recess 110 (FIG. 1B) to receive a barrel flange 112 of container 104. Barrel flange 112 within recess 110 can axially fix the position of container 104 relative to housing 102.

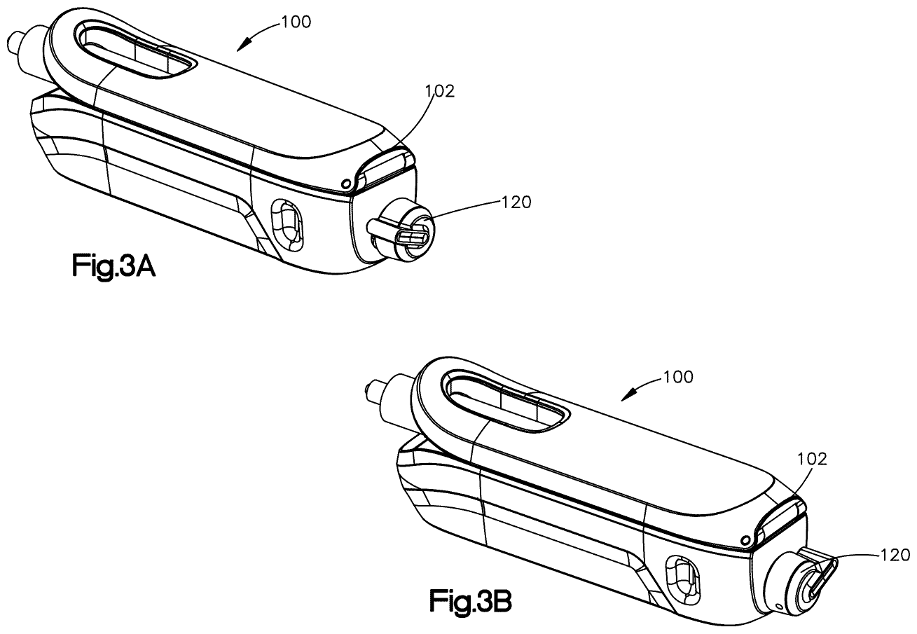

Referring to FIGS. 3A and 3B, a toggle 120 can be coupled to housing 102. Toggle 120 can be movable between a ready configuration (FIG. 3A) and a firing configuration (FIG. 3B). Injection aid 100 can apply a force to plunger 108 when toggle 120 is in the firing configuration. Injection aid 100 can be prevented from applying a force to plunger 108 when toggle 120 is in the ready configuration. Cap 116 can be moved between the open and closed position when toggle 120 is in the ready configuration. Toggle 120 can remain in the ready configuration as cap 116 is moved from the open position to the closed position such that an injection force is not automatically applied to the plunger 108 as the cap 116 moves to the closed position. Instead, the injection force is not applied to plunger 108 until toggle 120 moves to the firing configuration. This can allow a health care provider or user to prepare injection aid 100 for use by inserting container 104 into housing 102 and moving cap 116 to the closed position without the injection force being immediately applied to plunger 108. In alternative embodiments, moving cap 116 to the closed position moves toggle 120 to the firing configuration.

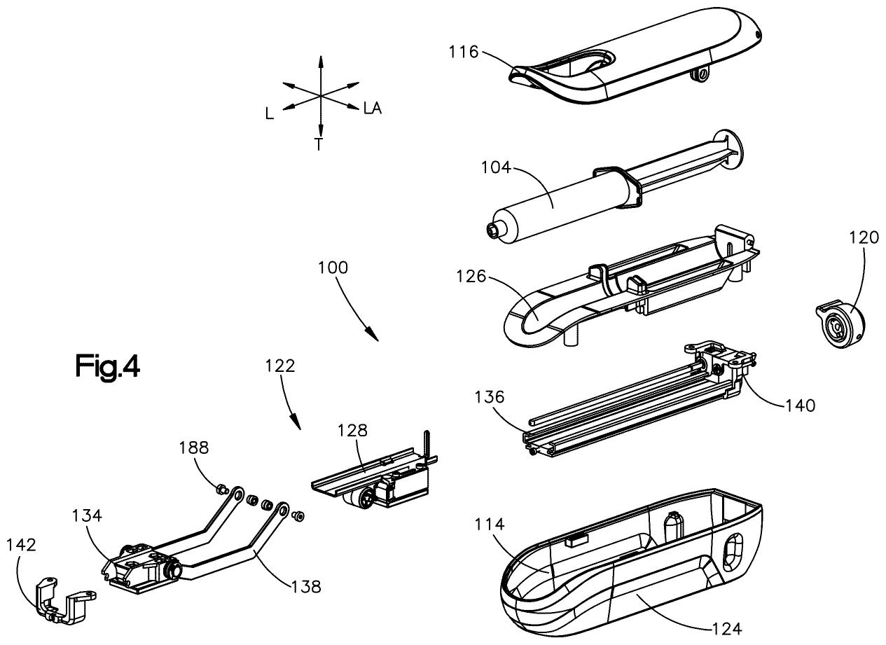

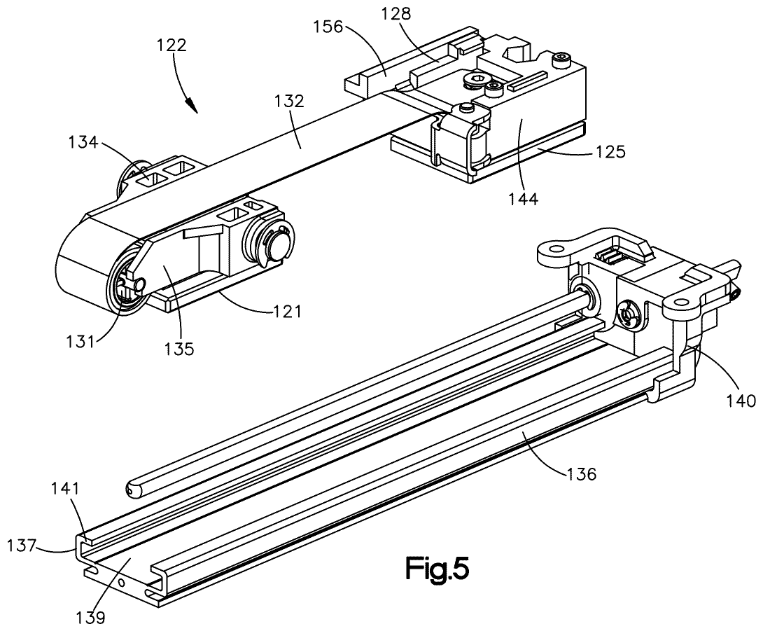

Referring to FIG. 4, injection device 100 can include an actuation assembly 122 adapted to apply the injection force to plunger 108. In some examples, actuation assembly 122 includes plunger rod 109 to apply the injection force to plunger 108. In other examples, container 104 includes plunger rod 109 and actuation assembly 122 engages plunger rod 109 to apply the injection force to plunger 108. Actuation assembly 122 can include a carriage 128 and an anchor 134. A linkage 138 can be coupled to each of anchor 134 and cap 116 such that the anchor 134 moves relative to housing 102 as cap 116 moves between the closed position and the open position. Anchor 134 can move relative to housing 102 as cap 116 moves between the open and closed positions. Anchor 134 can move toward carriage 128 as cap 116 moves from the closed position to the open position. Anchor 134 can move away from carriage 128 as cap 116 moves from the open position to the closed position. Anchor 134 can be fixed relative to housing 102 when the cap 116 is in the closed position. The carriage 128 can be coupled to anchor 134 by a biasing element 132 (FIG. 5). In some examples, carriage 128 can be adapted to contact plunger rod 109 to apply the injection force to plunger 108. In other examples, plunger rod 109 can be fixed to carriage 128. The injection force can be applied when cap 116 is in the closed position such that the biasing element 132 urges the carriage 128 toward anchor 134 to dispense medicament from container 104.

Actuation assembly 122 can be received by base 114. Base 114 can include a lower base 124 and an upper base 126 that define a recess to receive actuation assembly 122. Carriage 128 can be movable relative to barrel 106 of container 104. Carriage 128 can be movable from a first position to a second position along a carriage axis generally parallel to longitudinal axis L. Longitudinal axis L can be parallel to central axis A1. Longitudinal axis L can be perpendicular to a lateral axis LA. Transverse axis T can be perpendicular to each of longitudinal axis L and lateral axis LA.

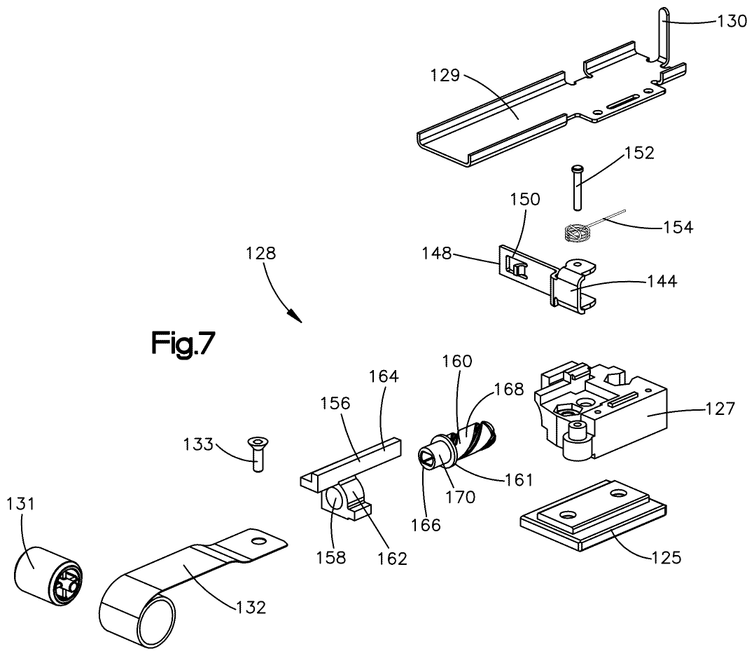

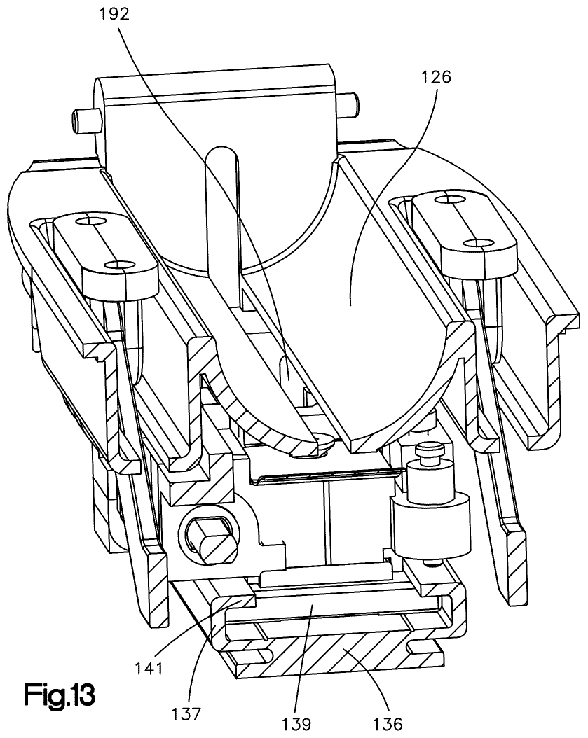

Referring to FIG. 7, carriage 128 can include a body 127 with a foot 125 adapted to engage a guide 136. Foot 125 can be coupled to body 127 via adhesive, fastener, or weld. In other embodiments, body 127 and foot 125 are a unitary construct. A deck 129 can be coupled to body 127. Deck 129 can have a length in longitudinal direction L that is longer than a length of body 127. Deck can be coupled to body 127 via adhesive, fastener, or weld. A tab 130 can extend from deck 129 and be adapted to contact and move plunger rod 109 as carriage 128 moves distally along the carriage axis. The upper base 126 can include a channel 192 (FIG. 13) such that tab 130 extends through the channel 192 from a first side of upper base 126 to a second side of upper base 126 opposite the first side. Deck 129 can prevent unintended access to internal mechanisms (e.g., anchor or carriage) through channel 192.

Referring to FIG. 7, a biasing element 132 can be adapted to move carriage 128 relative to housing 102. Biasing element 132 can be a constant force spring. Biasing element 132 can be adapted to provide a constant injection force throughout the injection. Biasing element 132 is shown in FIG. 7 as coupled to carriage 128 by a fastener 133 but could also be coupled via adhesive, weld, or interference fit. Biasing element 132 can be coupled to a drum 131. Biasing element 132 can be wound about a drum 131. Biasing element 132 can be configured to provide a force of about 10 Newtons (N) to about 20 N, about 20 N to about 30 N, about 30 N to about 40 N, about 40 N to about 50 N, about 50 N to about 60 N, about 60 N to about 70 N, or about 70 N to about 80 N. Biasing element 132 can be selected to provide a sustained force over a selected injection time period. Injection time period can be about 1 second to about 10 seconds, about 10 seconds to about 30 seconds, about 30 seconds to about 1 minute, about 1 minute to about 5 minutes, about 5 minutes to about 10 minutes, about 10 minutes to about 30 minutes, about 30 minutes to about 1 hour, or greater than about 1 hour.

Referring to FIG. 5, actuation assembly 122 can include an anchor 134 coupled to biasing element 132. Anchor 134 can include one or more arms 135 adapted to engage drum 131. Arms 135 can include a notch adapted to receive a protrusion or axle of drum 131 such that drum is rotatably coupled to arms 135. Anchor 134 can be fixed relative to housing 102 when the injection force is applied to plunger rod 109. In some embodiments, biasing element 132 is in tension between anchor 134 and carriage 128 such that biasing element 132 urges carriage 128 toward anchor 134. Carriage 128 and anchor 134 can each be coupled to guide 136. Anchor 134 can include a foot 121 adapted to engage guide 136. Foot 121 can be coupled to anchor 134 by a fastener 123. Alternatively, foot 121 can be coupled to anchor 134 by adhesive, weld, or anchor 134 and foot 121 can be monolithically formed.

Guide 136 can be fixed relative to housing 102. In some embodiments, guide 136 can define a recess to receive foot 121 and foot 125. In other embodiments, foot 121 and foot 125 can each be a recess and guide 136 can be a male track that is received by the recess of foot 121 and foot 125. Foot 121 and foot 125 can engage guide 136 to maintain alignment of carriage 128 and anchor 134 relative to housing 102. Referring to FIG. 5, guide 136 can include a sidewall 137 that extends away from a guide surface 139. Guide 136 can include a lip 141 coupled to sidewall 137. Lip 141 can at least partially extend over an upper surface of foot 125 and foot 121 to prevent foot 125 and foot 121 disengaging from guide 136. A proximal end cap 140 can be coupled to a proximal end of guide 136. A distal end cap 142 (FIG. 4) can be coupled to a distal end of guide 136. Proximal end cap 140 and distal end cap 142 can each be coupled to housing 102 via adhesive, weld, or fastener.

As discussed above, anchor 134 can be fixed relative to housing 102 when an injection force is applied to container 104. Anchor 134 can be movable relative to housing 102 when an injection force is not being applied. Anchor 134 can be movable relative to housing 102 as cap 116 moves between the open position and the closed position. Anchor 134 can be movable relative to guide 136 as cap 116 moves between the open and closed positions. Anchor 134 can move relative to guide 136 along an axis parallel to longitudinal axis L.

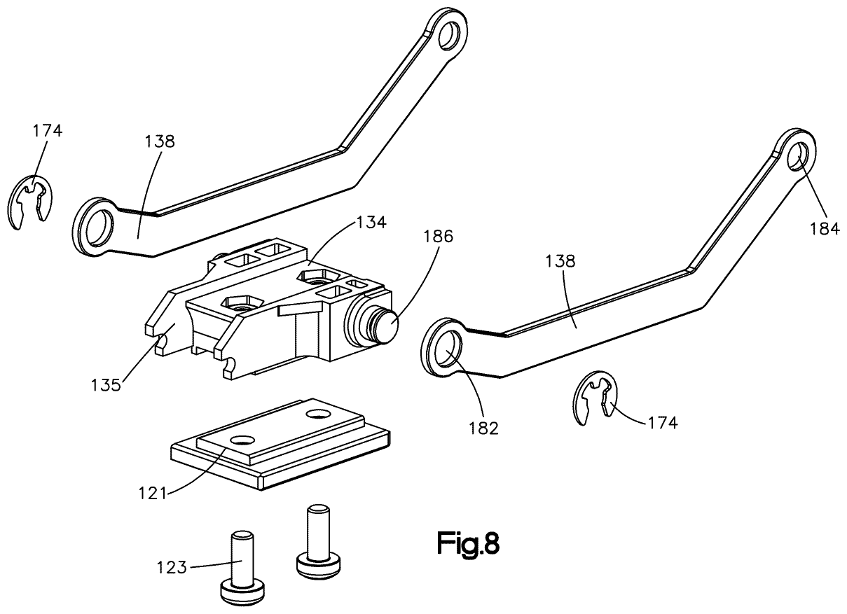

Referring to FIG. 8, a linkage 138 can be coupled to anchor 134 and to cap 116 such that cap 116 moves anchor 134 along guide 136 as cap 116 pivots between the closed position and the open position. Linkage 138 can move anchor 134 toward first end 103 of housing 102 as cap 116 transitions from the open position to the closed position. Linkage 138 can move anchor 134 toward second end 105 of housing 102 as cap 116 transitions from the open position to the closed position. Linkage 138 can be pivotably coupled to each of anchor 134 and cap 116. Linkage 138 can pivot relative to anchor 134 as cap 116 transitions between the open and closed positions. Linkage 138 can include a first end 182 and a second end 184 spaced from the first end 182 along a linkage central axis. First end 182 can be pivotably coupled to anchor 134. A fixation element 174 can couple linkage 138 to anchor 134. The fixation element 174 can be a retaining ring, fastener (e.g., threaded fastener), axel, rod, or dowel. First end 182 can include a recess to receive a protrusion 186 on anchor 134. The fixation element 174 can be adapted to engage protrusion 186 to rotatably couple linkage 138 to anchor 134. Protrusion 186 can extend through first end 182.

Linkage 138 can be pivotably coupled to cap 116. Second end 184 can be pivotably coupled to cap 116. Linkage 138 can pivot relative to cap 116 as cap 116 transitions between the open and closed positions. A fixation element 188 (FIG. 4) can pivotably couple second end 184 to cap 116. Fixation element 188 can be an axle, a dowel, a fastener (e.g., threaded fastener), or rod. Cap 116 can include a coupling element 190 adapted to couple to second end 184. Coupling element 190 can be a receiver adapted to receive fixation element 188. Alternatively, coupling element 190 can be a protrusion that is received by second end 184.

Linkage 138 can move anchor 134 toward carriage 128 as cap 116 moves from the closed position to the open position. Anchor 134 can move into contact with carriage 128 as linkage 138 moves anchor 134 longitudinally toward a proximal end of housing 102. Anchor 134 and carriage 128 can then move together toward proximal end of housing 102 as cap 116 continues to move toward the open position. In some examples, tab 130 of carriage 128 can disengage from plunger rod 109 as carriage 128 moves proximally relative to housing 102. In other examples, tab 130 is fixed to plunger rod 109 such that plunger rod 109 disengages from plunger 108 as carriage 128 moves proximally relative to housing 102. Tab 130 being disengaged from plunger 108, or plunger rod 109 being disengaged from plunger 108, can prevent dispensing medicament from container 104 when cap 116 is in the open position. This can also allow container 104 to be replaced when cap 116 is in the open position.

Linkage 138 can move anchor 134 along guide 136 to an injection position as cap 116 moves from the open position to the closed position. Anchor 134 can move a distance that is about 25%, about 50%, or about 75% of the length of the guide 136 as anchor 134 moves to the injection position. The injection position of the anchor 134 can be a distal most position of anchor 134. Anchor 134 can be spaced from distal end cap 142 when anchor 134 is in the injection position. In some embodiments, biasing element 132 applies a force to carriage 128 as anchor 134 moves relative to housing 102 such that carriage 128 and anchor 134 move together along the guide 136 as cap 116 moves from the open position to the closed position. For example, carriage 128 can move distally along guide 136 with anchor 134 until tab 130 moves into contact with plunger rod 109. In other examples, carriage 128 can move distally along guide 136 until plunger rod 109 contacts plunger 108. Plunger 108 can resist distal movement of carriage 128. Anchor 134 can continue to move relative to guide 136 after carriage 128 contacts plunger rod 109, or plunger rod 109 contacts plunger 108, such that anchor moves away from carriage 128 as cap 116 moves to the closed position. Movement of anchor 134 away from carriage 128 can increase tensile force of biasing element 132. Linkage 138 can maintain anchor 134 in the injection position when cap 116 is in the closed position.

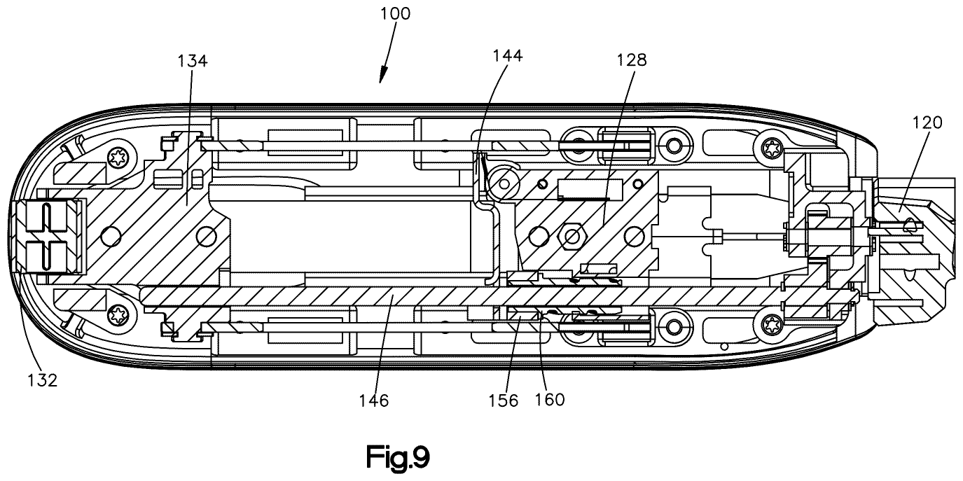

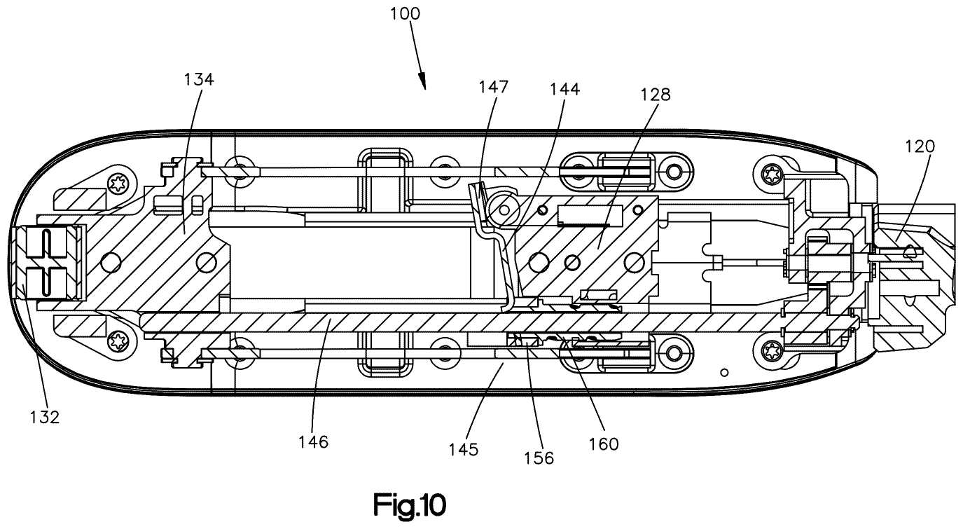

Biasing element 132 can have sufficient force to effectuate movement of plunger 108 when carriage 128 contacts plunger rod 109. Referring to FIGS. 9 and 10, injection aid 100 can include a brake assembly 145 to at least temporarily fix the position of carriage 128 relative to guide 136. Brake assembly 145 can prevent movement of carriage 128 when brake assembly is in an engaged position. Brake assembly 145 can allow movement of carriage 128 when brake assembly is in a released position. Brake assembly 145 can be in the engaged position as cap 116 moves from the open position toward the closed position to prevent dispensing of medicament before the brake assembly 145 is in the released position.

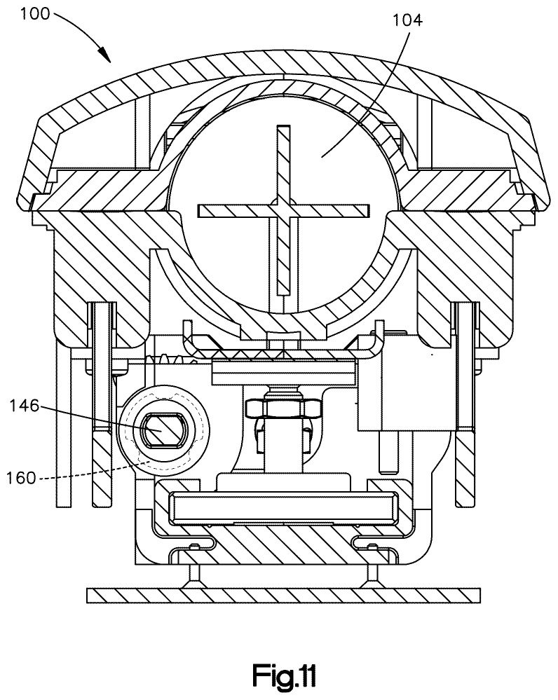

Referring to FIGS. 9 and 10, brake assembly 145 can include a latch 144 and a rod 146. Rod 146 can be axially fixed relative to proximal end cap 140. Rod 146 can be rotatable relative to proximal end cap 140. Rod 146 can include a first end and a second end spaced from the first end along a rod central axis. Rod central axis can be parallel to longitudinal axis L. At least a portion of rod 146 can have a keyed profile (FIG. 11) such that rod 146 can be engaged and rotated by a tool. The keyed profile can include a non-circular cross-sectional shape when taken along a plane that extends in the transverse direction T and the lateral direction LA. For example, rod 146 can have an oblong cross-sectional shape. In some embodiments, the keyed profile of rod 146 extends at least half the length of rod 146 in the longitudinal direction.

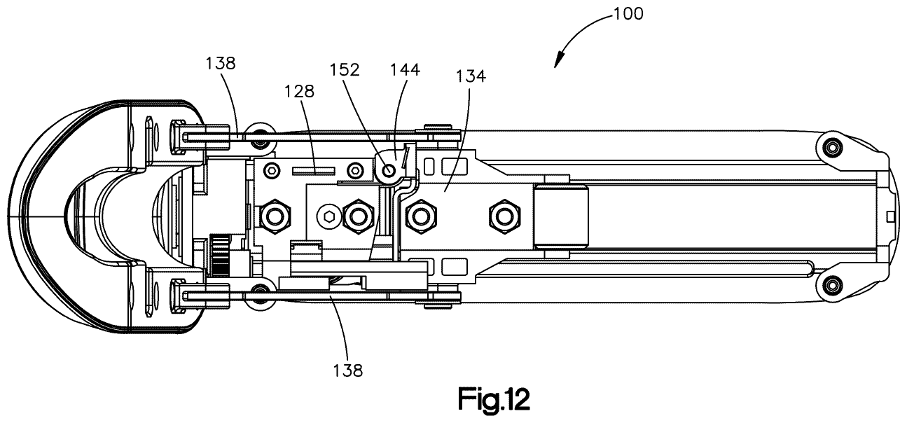

Latch 144 can be movable relative to rod 146. Latch 144 can be coupled to carriage 128 such that latch 144 moves axially with carriage 128. Latch 144 can be axially moveable relative to rod 146 along an axis parallel to longitudinal axis L when brake assembly is in a disengaged position. Latch 144 can include an opening 148 to receive rod 146. Rod 146 can extend through an opening in each of latch 144 and carriage 128. Opening 148 can be defined by a sidewall 150. Latch 144 can engage rod 146 when brake assembly is in the engaged position. For example, sidewall 150 can engage rod 146 such that latch 144 is fixed relative to rod 146 when latch 144 is in the engaged position. Sidewall 150 of latch 144 can be disengaged from rod 146 when latch 144 is in the disengaged position. Latch 144 can be axially fixed relative to rod 146 when brake assembly 145 is in an engaged position. Latch 144 can be pivotably coupled to carriage 128. Latch 44 can include a first end and a second end opposite the first end. The first end can be pivotably coupled to carriage 128. The second end can be a free end. Latch 144 can be moveable relative to rod 146 along an arcuate path as brake assembly 145 moves between the released position and the engaged position. Latch 144 can pivot about an axis 152 (FIG. 12) between the engaged position (FIG. 9) and the released position (FIG. 10).

Latch 144 can engage rod 146 in the engaged position. Latch 144 can be biased toward the engaged position. A biasing element 154 can bias latch 144 toward the engaged position. In some embodiments, biasing element 154 is a spring. In other embodiments, biasing element 154 is a resilient material such as rubber.

Brake assembly 145 can include a runner 156 adapted to move latch 144 between the engaged position and the released position. For example, runner 156 can move axially into contact with latch 144. Continued axial movement of runner 156 after contacting latch 144 can cause latch 144 to pivot about axis 152. Runner 156 can move latch 144 distally.

Runner 156 can include a beam 164 adapted to contact anchor 134. Beam 164 can have an L-shape in cross-section. The L-shape can provide increased stiffness compared to a beam with a rectangular cross-section. Beam 164 can extend distally beyond a distal end of a body 162 of runner 156. Beam 164 can extend distally beyond a distal end of latch 144 when latch 144 is in the engaged position, the released position, or both the engaged position and the released position.

Brake assembly 145 can include an actuator 160 adapted to engage body 162. Runner 156 can include an opening 158 in body 162 to receive a portion of actuator 160. Actuator 160 can be rotatable relative to body 162 when actuator is in opening 158. Actuator 160 being rotatable relative to body 162 when actuator 160 is in opening can prevent rotary motion of actuator 160 from being transferred to latch 144. In other embodiments, runner 156 and actuator 160 are a unitary construct. Body 162 of runner 156 and actuator 160 can be positioned between carriage body 127 and latch 144. Actuator 160 can include an opening 166 to receive rod 146. Rod 146 can extend through openings in each of carriage 128, latch 144, runner 156, and actuator 160. Actuator 160 can translate axially relative to rod 146 while rod 146 is in opening 166. Opening 166 of actuator 160 can be keyed to the shape of rod 146 such that rotation of rod 146 about the rod longitudinal axis cause actuator 160 to rotate. Alternatively, actuator 160 can include a set screw that rotationally fixes actuator 160 relative to rod 146.

Actuator 160 can be adapted to move latch 144 from the engaged position to the released position. Actuator 160 can be movable relative to latch 144 and carriage body 127. Actuator 160 can move between a first position (FIG. 9) and a second position (FIG. 10). Actuator 160 can be in the first position when latch 144 is in the released position. Actuator 160 can be in the second position when latch 144 is in the engaged position. Actuator 160 can include a first portion 168 and a second portion 170 separated by a flange 161. Flange 161 can have a maximum cross-sectional length that is greater than a maximum cross-sectional length of at least one of first portion 168 and second portion 170. Actuator 160 can include threads adapted to engage an opening in body 127 of carriage 128. The threaded engagement of actuator 160 and carriage body 127 can cause axial movement of actuator 160 as actuator rotates. Actuator 160 can be rotationally fixed relative to rod 146 and axially movable relative to rod 146. First portion 168 can include threads to threadedly engage carriage body 127. Second portion 170 can be received by opening 158 of runner 156. Flange 161 can engage runner 156 when second portion 170 is in opening 158 such that flange 161 engages runner 156. Alternatively, an end surface of actuator 160 can engage runner 156 such that runner 156 can move actuator 160 relative to carriage body 127. Actuator 160 can move distally relative to housing 102 as actuator rotates such that actuator moves runner 156 into engagement with latch 144 and moves latch 144 to the disengaged position.

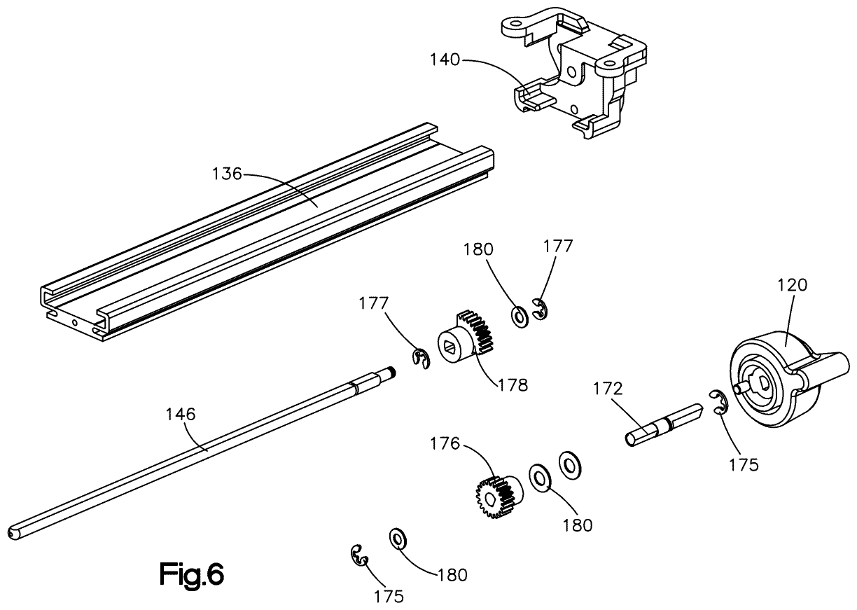

Toggle 120 can be adapted to move actuator 160. Toggle 120 can be rotationally fixed relative to rod 146 such that rod 146 rotates as toggle 120 rotates. Toggle 120 can be keyed to the shape of rod 146 such that toggle 120 is rotationally fixed relative to rod 146. Toggle 120 can be positioned on an exterior side of proximal end cap 140. Rod 146 can be positioned on an interior side of proximal end cap 140. In some embodiments, rod 146 extends from interior side of proximal end cap 140 to exterior side of proximal end cap 140 and toggle 120 is coupled directly to rod 146. In other embodiments, toggle 120 is coupled to a shaft 172 (FIG. 6). Shaft 172 can extend from the exterior side to the interior side of proximal end cap 140. Shaft 172 can have a longitudinal length that is greater than a width of proximal end cap 140 along longitudinal axis L. The position of shaft 172 along longitudinal axis L can be fixed. For example, one or more fixation elements 175 (e.g., a retaining ring or set screw) can be coupled to the shaft 172 to fix the position of shaft 172 along longitudinal axis L. Shaft 172 can be coupled to a gear 176. Gear 176 can be a spur gear. A spacer 180 can be positioned between proximal end cap 140 and gear 176. Spacer 180 can be a washer, a bushing, etc. A first fixation element 175 can be coupled to shaft 172 on a first side of gear 176 and a second fixation element 175 can be coupled to shaft 172 on second side of gear 176 opposite the first side.

A rod gear 178 can be coupled to rod 146. The position of rod gear 178 along the length of rod 146 can be fixed. A fixation element 177 (e.g., a retaining ring or set screw) can be coupled to rod 146. Fixation element 177 can fix the position of rod gear 178 relative to rod 146. A first fixation element 177 can be coupled to rod 146 on a first side of rod gear 178 and a second fixation element 177 can be coupled to rod 146 on a second side of rod gear 178. The first side can be opposite the second side along the length of rod 146. Rod gear 178 can be rotationally fixed relative to rod 146. Rod gear 178 can be adapted to mesh with gear 176 such that rod 146 rotates as toggle 120 rotates. Rod gear 178 can be a sector gear. Toggle 120 can be rotated in a first direction from the firing configuration to the ready configuration while medicament is being dispensed to pause the dispensing. Toggle 120 can be rotated about an axis generally parallel to longitudinal axis L as toggle rotates in the first direction. Toggle 120 can be rotated in a second direction opposite the first direction from the ready configuration to the firing configuration to resume the dispensing. Injection aid 100 can be prevented from dispensing medicament after cap 116 is closed until toggle 120 is moved from the ready configuration to the firing configuration. Toggle 120 can also be in the ready configuration as cap 116 is moved from the open position to the closed position such that the injection force is applied to plunger 108 upon engagement of carriage 128 with plunger rod 109 or engagement of plunger rod 109 with plunger 108.

Movement of cap 116 from the closed position to the open position can move latch 144 from the engaged position to the released position. Latch 144 can include a tab 147 (FIG. 10) adapted to be engaged by anchor 134. Anchor 134 can move proximally relative to carriage 128 as cap 116 moves from the closed position to the open position as previously described. Anchor 134 can engage tab 147 and move latch 144 from the engaged position to the released position as anchor 134 moves in response to opening cap 116. Carriage 128 can move distally relative to housing 102 as medicament is dispensed. Tab 147 can move into engagement with anchor 134 as an injection is completed.

Anchor 134 can be movable relative to housing 102 while latch 144 is in the engaged position. Carriage 128 can be fixed relative to housing 102 during an initial movement of anchor 134. Actuator 160 can be in either the first position or the second position as anchor 134 moves relative to carriage 128.

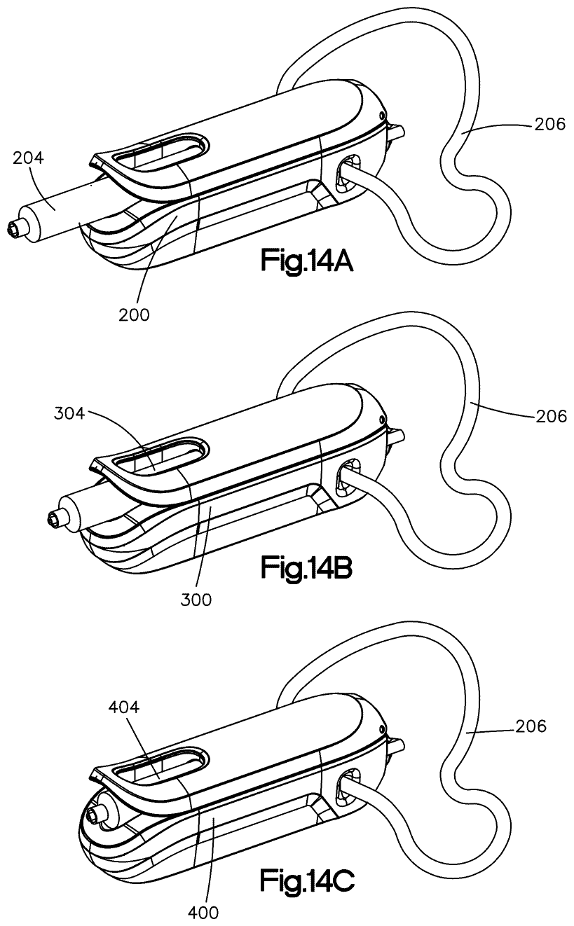

FIGS. 14A to 14C show additional embodiments of an injection aid (e.g., 200, 300, 400). Injection aid (e.g., 200, 300, 400) can be adapted to receive different size containers (e.g., 204, 304, 404). Injection aid (e.g., 200, 300, 400) can include a passageway between the cap and base even when cap is in the closed position such that a portion of container (e.g., 204, 304, 404) extends distally beyond a distal end of cap and base. Injection aid (e.g., 200, 300, 400) can include a retainer 206 such that injection aid (e.g., 200, 300, 400) can be coupled to a user. Retainer 206 can be a string, adjustable belt, chain, or pin.

It should be noted that the illustrations and discussions of the embodiments and examples shown in the figures are for exemplary purposes only and should not be construed limiting the disclosure. One skilled in the art will appreciate that the present disclosure contemplates a range of possible modifications of the various aspects, embodiments and examples described herein. Additionally, it should be understood that the concepts described above with the above-described embodiments and examples may be employed alone or in combination with any of the other embodiments and examples described above. It should further be appreciated that the various alternatives described above with respect to one illustrated embodiment can apply to all other embodiments and examples described herein, unless otherwise indicated. Reference is therefore made to the claims.

1. An injection aid comprising:
a housing adapted to receive a container including a medicament chamber;
an actuation assembly adapted to move a plunger relative to the medicament chamber to dispense medicament out of the medicament chamber; and
a toggle movable from a ready configuration to a firing configuration whereby the actuation assembly urges the plunger to move, the toggle movable from the firing configuration to the ready configuration whereby the plunger is fixed relative to the housing to pause dispensing medicament, and the toggle moveable from the ready configuration to the firing configuration to resume dispensing medicament from the medicament chamber.

2. The injection aid of claim 0, wherein the housing includes a first end and a second end spaced from the first end along a central axis, and the actuation assembly includes a carriage movable relative to the housing along an axis parallel to the central axis and the carriage is engageable with the plunger to move the plunger relative to the container.

3. The injection aid of claim 2, further comprising a brake assembly adapted to arrest movement of the carriage, wherein the toggle moves the brake assembly from an engaged position to a released position as the toggle moves between the ready configuration and the firing configuration.

4. The injection aid of claim 3, wherein the brake includes:
a rod having a first end and a second end spaced from the first end along a rod axis parallel to the central axis; and
a latch having an opening defined by a sidewall, the rod extending through the opening in the latch, wherein the sidewall engages the rod such that the rod is axially fixed relative to the latch when the brake assembly is in the engaged position and the rod is disengaged from the sidewall such that the latch is axially movable relative to the rod when the brake assembly is in the released position.

5. The injection aid of claim 4, wherein the toggle rotates the rod about the rod axis as the toggle moves from the ready configuration to the firing configuration.

6. The injection aid of claim 4, wherein the actuation assembly includes an anchor movable relative to the carriage and the housing, and
wherein the housing includes a base, a cap pivotably coupled to the base, and a linkage coupled to the cap and the anchor such that pivotal movement of the cap relative to the base axially moves the anchor relative to the housing.

7. The injection aid of claim 6, wherein the cap is pivotable relative to the base when the toggle is in either of the ready configuration or the firing configuration.

8. The injection aid of claim 6, wherein pivotal movement of the cap relative to the base axially moves the anchor and the carriage relative to the base.

9. The injection aid of claim 8, wherein the pivotal movement of the cap relative to the base moves the anchor a first distance and moves the carriage a second distance less than the first distance.

10. The injection aid of claim 6, wherein the carriage engages the plunger as the cap moves from an open position to a closed position.

11. The injection aid of claim 6, wherein the actuation assembly includes a biasing element that provides a force against the carriage, thereby urging the plunger to move relative to the medicament chamber, wherein the anchor is coupled to the carriage by the biasing element.

12. The injection aid of claim 11, wherein the biasing element comprises a constant force spring.

13. The injection aid of claim 12, further comprising a rail within the housing, the carriage and the anchor each moveable along the rail.

14. The injection aid of claim wherein the container is removably coupled to the housing and the injection aid is adapted to be reusable.

15. A method of dispensing medicament from an injection aid including a housing with a cap and a base, and a container within the housing, the container including a medicament chamber adapted to hold medicament, the method comprising:
moving the cap relative to the base from an open position to a closed position such that a carriage engages a plunger rod;
activating a toggle from a ready configuration to a firing configuration; and
applying a force to the plunger rod when the toggle is in the firing configuration to dispense medicament out of the medicament chamber.

16. The method of claim 15, further comprising moving the toggle from the injection configuration to the ready configuration while medicament is being dispensed from the container to pause the dispensing.

17. The method of claim 16, further comprising moving the toggle from the ready configuration to the injection configuration to resume dispensing the medicament from the container.

18. The method of claim 15, further comprising moving the toggle from the injection configuration to the ready configuration;
moving the cap relative to the base from the closed position to the open position such that the carriage disengages from the plunger rod; and
removing the container from the housing.

19. The method of claim 18, further comprising inserting a replacement container into the housing.

20. The method of claim 15, wherein activating the toggle includes disengaging a brake assembly such that the carriage is moveable relative to the container.
